(19) United States
(12) Patent Application Publication
(10) Pub. No.: US 2010/0331879 A1
(43) Pub. Date: Dec. 30, 2010

(54) Articulating Surgical Hand Tool

(75) Inventors: Anthony N. Harris, Columbia, MO (US); Jonathan R. Thompson, Cincinnati, OH (US); Rebecca J. Rone, Columbia, MO (US)

Correspondence Address: D''Hue Law, PO Box 421972, Indianapolis, IN, 46242-1972, US

(73) Assignee: THE CURATORS OF THE UNIVERSITY OF MISSOURI, Columbia, MO (US)

(21) Appl. No.: 12/824,127

(22) Filed: Jun. 25, 2010

Related U.S. Application Data
(60) Provisional application No. 61/269,497, filed on Jun. 25, 2009.
(60) Provisional application No. 61/279,917, filed on Oct. 28, 2009.

(51) Int. Cl. A61B 17/28

(52) U.S. Cl. 606/205

(57) ABSTRACT

A surgical instrument comprising a frame, a control-effector coupled to the frame, a shaft, and an end effector in fluid communication with the control-effector is disclosed.

### CROSS REFERENCE TO RELATED APPLICATION

This application claims the benefit of U.S. Provisional Patent Application No. 61/269,497, filed Jun. 25, 2009, the contents of which are expressly incorporated by reference. This application also claims the benefit of U.S. Provisional Patent Application No. 61/279,917, filed Oct. 28, 2009, the contents of which are expressly incorporated by reference.

### FIELD

The field of this disclosure relates to the use of hydraulic actuation to transmit direct human force on a control-effector to an end effector.

### BACKGROUND

The history and evolution of laparoscopic surgery has spanned the last twenty-five (25) years. Advances in surgery have transitioned from open techniques to less invasive procedures and techniques. This occurrence has given rise to many new innovations in surgical tools used in the operating room, imaging suites and at the bedside. The clinical advantages of less invasive techniques in the surgical treatment of diseases have been well documented. A growing list of advantages of beneficial attributes of minimally invasive surgery (MIS) include decreases in morbidity, mortality, patient recovery-time, operating room time, and patient pain.

MIS surgical instruments include an end effector, a control effector, and a shaft which extends between the end effector and the control effector. The end effector is the portion of the instrument configured to engage tissue of the patient to perform a surgical procedure. The end effector and the shaft are shaped for insertion through a small incision on the patient. Typically a trocar (and/or cannula) is maintained at the incision to aid in insertion of the surgical instrument. The shaft tends to be elongated to allow for the end effector to reach tissue of the patient.

The shaft also allows for adjustment in positioning and/or orientation of the end effector. Articulation is conventionally described as transverse or non-axial movement of the end effector relative to the shaft. Articulation allows the end effector to reach and/or engage tissue from a plurality of angles and orientations. Articulation also allows for the end effector to maneuver around obstacles to reach the surgical objective. MIS surgical instruments benefit from increased articulation.

The recent advent of robotic assisted surgery (RAS) has enabled surgeons to expand their technique and usefulness of MIS approaches. RAS enables less technically skilled laparoscopists the ability to perform traditionally difficult procedures in record times. RAS advantages are accomplished through robotically enhanced dexterity and intuitive control of an end effector used for intraoperative tissue manipulation. Particularly, the at least six (6) degrees of freedom capability of robotic surgery has been a boon to procedures which are difficult and time-consuming to perform with traditional non-robot assisted surgical tool which typically have only five (5) degrees of freedom.

### SUMMARY

The present disclosure includes a surgical instrument comprising a frame, a shaft coupled to the frame, the shaft sized to pass through a trocar, the shaft conformable into a plurality of orientations, an end effector coupled to the shaft, the end effector sized to pass through the trocar, the end effector and shaft providing at least six degrees of freedom to the end effector relative to the frame, and a hydraulic articulation control system including a control-effector and at least one bellow, the at least one bellow coupled to the control-effector and the end effector, the at least one bellow used to transfer hydraulic force from the control-effector to the end effector.

The present disclosure also includes a surgical instrument comprising a frame, a shaft including a proximal end coupled to the frame, the shaft sized to pass through a cannula of a trocar, wherein the shaft is bendable along its longitudinal axis, a end effector coupled to the shaft, the end effector sized to pass through the cannula of the trocar, the end effector including a pitch joint and a yaw joint, the pitch joint providing rotation of the end effector relative to the shaft in a tilt forward or tilt backward motion, the yaw joint providing rotation of the end effector relative to the shaft in a turn left or turn right motion, the shaft including a roll joint, the roll joint providing rotation of the end effector relative to the frame in a tilt side to side motion, and a hydraulic articulation control system including a control-effector disposed within the frame, and at least one bellow in hydraulic communication with the control-effector, the at least one bellow used to transfer force from the control-effector to the end effector.

The present disclosure also includes a method of operating a surgical instrument, wherein the surgical instrument comprises a frame, a shaft coupled to the frame, a hydraulic control system including a control-effector, an end effector, and at least one bellow in fluid communication with the control-effector, the at least one bellow in mechanical connection with the end effector, the method comprising the steps of applying a force to the control-effector, transferring hydraulic fluid from the control-effector to the at least one bellow, transferring force from the at least one bellow to the end effector, and causing at least a portion of the end effector to rotate along at least one of a pitch joint or a yaw joint.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 depicts a perspective view of the surgical instrument according to one embodiment of the present disclosure.

FIG. 2 depicts a perspective view of the end effector of the surgical instrument of FIG. 1 according to one embodiment of the present disclosure.

FIG. 3 depicts a perspective view of the end effector of the surgical instrument of FIG. 1 according to one embodiment of the present disclosure. The end effector is shown with the shaft of the surgical instrument of FIG. 1 made transparent to illustrate a portion of the hydraulic actuation system including at least one bellow. The end effector is illustrated in a neutral orientation.

FIG. 4A illustrates an exploded view of at least one bellow of FIG. 3 according to a first embodiment of the present disclosure.

FIG. 4B illustrates an exploded view of at least one bellow of FIG. 3 according to a first embodiment of the present disclosure.

FIG. 4C illustrates an exploded view of at least one bellow according to a second embodiment of the present disclosure.

FIG. 4D illustrates an exploded view of at least one bellow of FIG. 4C according to a second embodiment of the present disclosure.

FIG. 4E1 illustrates a perspective view with a housing of the hydraulic system removed to illustrate at least one bellow of FIG. 4D according to a second embodiment of the present disclosure. The at least one bellow is illustrated in an extended orientation.

FIG. 4E2 illustrates a perspective view with a housing of the hydraulic system removed to illustrate at least one bellow of FIG. 4D according to a second embodiment of the present disclosure. The at least one bellow is illustrated in a retracted orientation.

FIG. 4F illustrates an exploded view of at least one bellow according to a third embodiment of the present disclosure.

FIG. 4G illustrates an exploded view of at least one bellow according to a third embodiment of the present disclosure.

FIG. 4H illustrates an exploded view of at least one bellow according to a fourth embodiment of the present disclosure.

FIG. 4I illustrates an exploded view of at least one bellow according to a fourth embodiment of the present disclosure.

FIG. 4J1 illustrates a perspective view of the at least one bellow of FIG. 4I according to a fourth embodiment of the present disclosure. The at least one bellow is illustrated in an extended orientation.

FIG. 4J2 illustrates a perspective view of the at least one bellow of FIG. 4I according to a fourth embodiment of the present disclosure. The at least one bellow is illustrated in a retracted orientation.

FIG. 5 depicts a perspective view of the end effector of the surgical instrument of FIG. 1 according to one embodiment of the present disclosure. The end effector is shown with a portion of the housing of the end effector of FIG. 1 made transparent to illustrate a portion of the hydraulic actuation system including at least one bellow. The end effector is illustrated in a neutral orientation.

Corresponding reference characters indicate corresponding parts throughout the several views. Although the drawings represent embodiments of the present disclosure, the drawings are not necessarily to scale and certain features may be exaggerated in order to better illustrate and explain the present disclosure.

### DETAILED DESCRIPTION OF THE EXEMPLARY EMBODIMENTS

The embodiments disclosed below are not intended to be exhaustive or limit the disclosure to the precise forms disclosed in the following detailed description. Rather, the embodiments are chosen and described so that others skilled in the art may utilize their teachings.

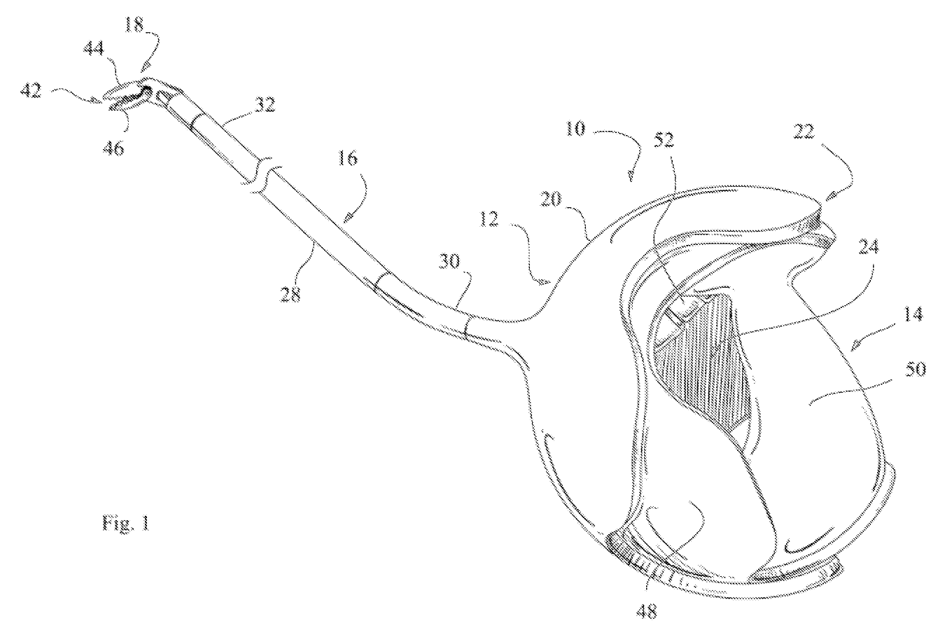

FIG. 1 illustrates surgical instrument 10 according to an embodiment of the present disclosure. As used herein, the term “surgical instrument” means a surgical hand tool used in human and animal MIS procedures as part of RAS. During operation, an operator, typically a surgeon, inserts surgical instrument 10 into the body of a human or animal (hereinafter described as “patient”). Surgical instrument 10 is used to manipulate tissue of the patient during MIS procedure as part of RAS. Surgical instrument 10 is a three dimensional singular body. Movement of surgical instrument 10 in three degrees of freedom: heaving (i.e., moving up and down), swaying (moving left and right), and surging (moving forward and backward) is directly translated to all parts of surgical instrument 10.

Surgical instrument 10 includes frame 12, control effector 14, shaft 16, and end effector 18. Frame 12 includes frame body 20. In this illustrative embodiment, frame body 20 has a general spherical shape. Frame body 20 defines frame body opening 22 and frame body cavity 24. Portions of control effector 14 are located within frame body cavity 24. Frame body opening 22 provides an operator (not shown) of surgical instrument 10 access to portions of control effector 14.

Frame 12 also includes frame projection 26 which couples to shaft 16. Frame projection 26 defines a portion of frame body cavity 24. Frame projection 26 provides fluid communication through frame 12.

Shaft 16 is coupled to frame 12 and end effector 18. Shaft 16 includes shaft housing 28. Shaft 16 and shaft housing 28 are each sized to pass through a trocar (not shown) or a cannula (not shown). Shaft 16, shaft housing 28, and end effector 18 are each elongated to allow end effector 18 to reach several parts of the patient.

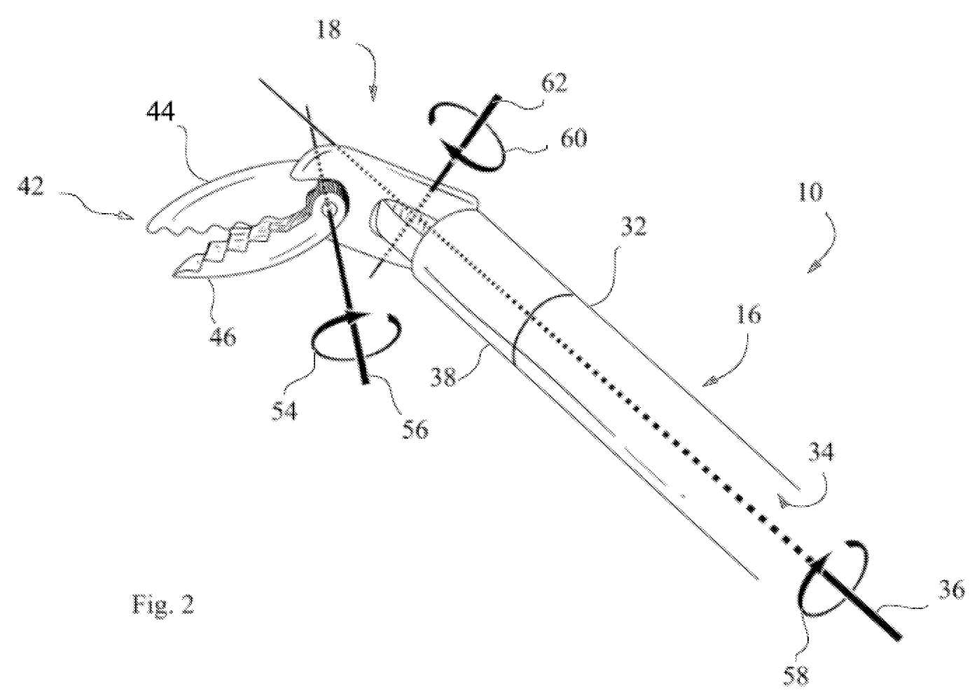

Proximal end 30 of shaft housing 28 couples to frame projection 26. Distal end 32 of shaft housing 28 couples to end effector 18. As used herein, the terms “proximal” and “distal” are measured in reference to the operator of surgical instrument 10. In most orientations, the operator is envisioned as positioned closer to frame 12 than shaft 16. Shaft housing 28 defines shaft cavity 34 (FIG. 2) which is in fluid communication with frame body cavity 24.

Frame projection 26 and proximal end 30 of shaft 16 provide fluid communication between frame body cavity 24 and shaft cavity 34 (FIG. 2). As used herein, the term “fluid communication” means that there is fluid in communication between parts of surgical instrument 10. In this embodiment, there is an opening sized to permit passage of any fluid (liquid or gas), for example hydraulic fluid, between the frame body 20 and shaft cavity 34. As used herein, the term “hydraulic fluid” refers to any fluid suitable for use in a hydraulic system, such as oil, air, biocompatible fluids such as saline and glycerin oil.

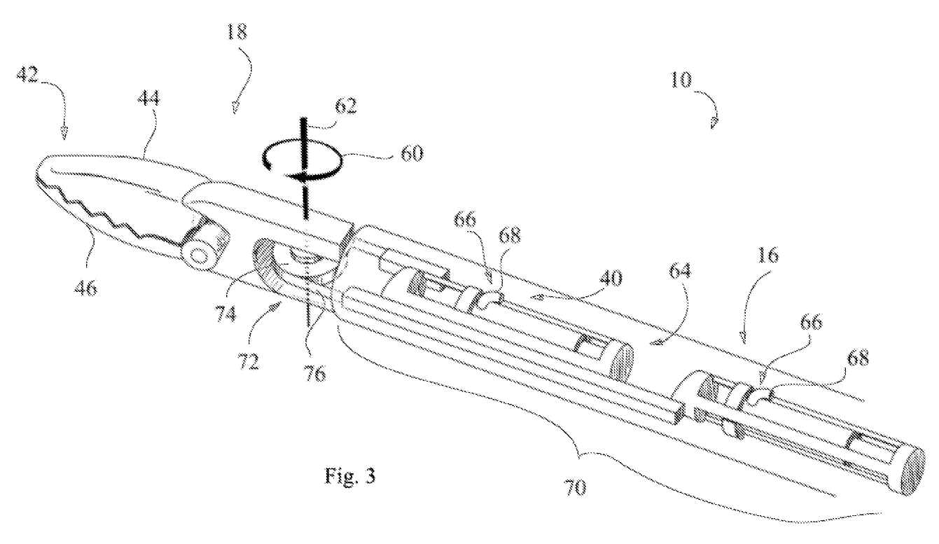

As best illustrated in FIG. 2, shaft 16 defines shaft cavity 34. Shaft cavity 34 extends along longitudinal axis 36 of shaft 16. Distal end 32 of shaft 16 and proximal end 38 of end effector 18 provide fluid communication between shaft cavity 34 and end effector cavity 40 (FIG. 3). In this embodiment, there is an opening sized to permit passage of any fluid (liquid or gas), for example hydraulic fluid, between the frame body 20 and shaft cavity 34.

Shaft 16 is also conformable into a plurality of orientations (i.e. bendable along longitudinal axis 36) either prior to use or during operation of surgical instrument 10. As used herein, the term “active contour” is used to describe this conformable nature of shaft 16. Shaft 16 can be composed of an elastomeric substance, such as plastic or rubber, a metallic compound, a synthetic substance, such as nylon, carbon fiber, or carbon nanotube fabric, or combinations thereof. Shaft 16 provides articulation of end effector 18. As used herein, the term “articulation” means transverse or non-axial movement of end effector 18 relative to shaft 16. As previously stated, articulation allows end effector 18 to reach and/or engage tissue from a plurality of angles and orientations. Articulation also allows for end effector 18 to maneuver around obstacles to reach the surgical objective.

As used herein, end effector 18 illustrates clamp mechanism 42. More specifically, clamp mechanism 42 is shown as a pair of jaw members 44, 46. However it will be appreciated that various embodiments of end effector 18 may be used for other surgical operations such as cutting, severing, stapling, grasping. End effector 18 may include various appendages such as clip appliers, access devices, drug/gene therapy delivery devices and/or laser energy devices. Furthermore, end effector 18 may include systems useful in endoscopy, ultrasound, and radio frequency.

Control effector 14 is slidably mounted to frame 12 such that control effector 14 can move in at least three degrees of freedom in relation to frame 12. Control effector 14 includes control body 48 which also has a generally spherical shape. Control body 48 is configured to correspond to the interior contour of frame body 20. Control effector 14 also includes handle 50. During operation, operator grasps handle 50 of surgical instrument 10. After grasping handle 50, the operator's hand and wrist motions move in concert with movement of end effector 18 in at least three degrees of freedom: pitch (i.e., tilting forward and backward), roll (i.e., tilting side to side), and yaw (i.e., turning left and right). Handle 50 includes lever 52 for actuation of at least one jaw member 46 of end effector 18 relative to the other jaw member 44 of end effector 18.

As illustrated in FIG. 2 and as described in greater detail below as well as in U.S. provisional patent application Ser. No. 61/279,917, filed Oct. 28, 2009, control effector 14 controls movement of end effector 18. End effector 18 is mounted to shaft 16 such that end effector 18 can move in at least three degrees of freedom in relation to frame 12. Based on the operator's rotation of control effector 14, shaft 16 provides rotation of end effector 18 about longitudinal axis 36 of shaft 16. Pitch arrow 54 illustrates rotation of end effector 18 about horizontal axis 56. Pitch arrow 54 illustrates rotation in a tilting forward and backward mode relative to frame 12. Roll arrow 58 illustrates rotation of end effector 18 about longitudinal axis 36. Roll arrow 58 illustrates rotation in a tilt side to side motion relative to frame 12. Yaw arrow 60 illustrates rotation of end effector 18 about vertical axis 62. Yaw arrow 60 illustrates rotation in a turning left and right orientation relative to frame 12. As previously described, the operator's hand and wrist motions control movement of end effector 18 in these at least three degrees of freedom: pitch (i.e., tilting forward and backward), roll (i.e., tilting side to side), and yaw (i.e., turning left and right).

As illustrated in FIGS. 3-5, surgical instrument 10 also includes hydraulic actuation system 64. Control effector 14 utilizes hydraulic actuation system 64 as a mechanism to control movement of end effector 18. Hydraulic actuation system 64 includes fluid wires 66 in communication with control effector 14. Fluid wires 66 are, among other things, fluid-filled closed and hermetically sealed systems. Each fluid wire 66 is not limited by length. The length of each fluid wire 66 may vary greatly, such as from approximately two (2) centimeters to approximately fifty (50) centimeters. The length of each fluid wire 66 may be directly proportional to the speed (i.e. flow and/or pressure) of compressive force transmitted from control effector 14 to end effector 18. The diameter of each tube 68 of fluid wire 66 may vary significantly and may be directly or indirectly proportional to the speed (i.e. flow and/or pressure) of compressive force transmitted from control effector 14 to end effector 18. Wall thickness of each tube 68 of fluid wire 66 may vary significantly and may be directly proportional to the amount of applied force necessary in order to cause movement at end effector 18.

Each fluid wire 66 has two points of action: (1) at least one proximal compression segment (not shown) in connection with control effector 14 and (2) at least one distal actuation segment 70 in mechanical connection with end effector 18. Control effector 14 transmits control of movement over end effector 18 by use of operator's compressive force upon proximal compression area (not shown). During operation of surgical instrument 10, operator exerts force on control effector 14. Proximal compression area (not shown) transmits operator's force to at least one distal actuation segment 70.

Distal actuation segment 70 is illustrated in FIG. 3 as bellow system 70. Please note that bellow system 70 is not limited to distal actuation segment 70. It is envisioned that bellow system 70 is also utilized as at least part of proximal compression area of control effector 14. Bellow system 70 undergoes conformational change in shape upon transmission of operator force from proximal compression area (not shown). The conformational change in shape is shown as either expansion (i.e. extension of bellow 84) or retraction (i.e. reduction, crumpling of bellow 84).

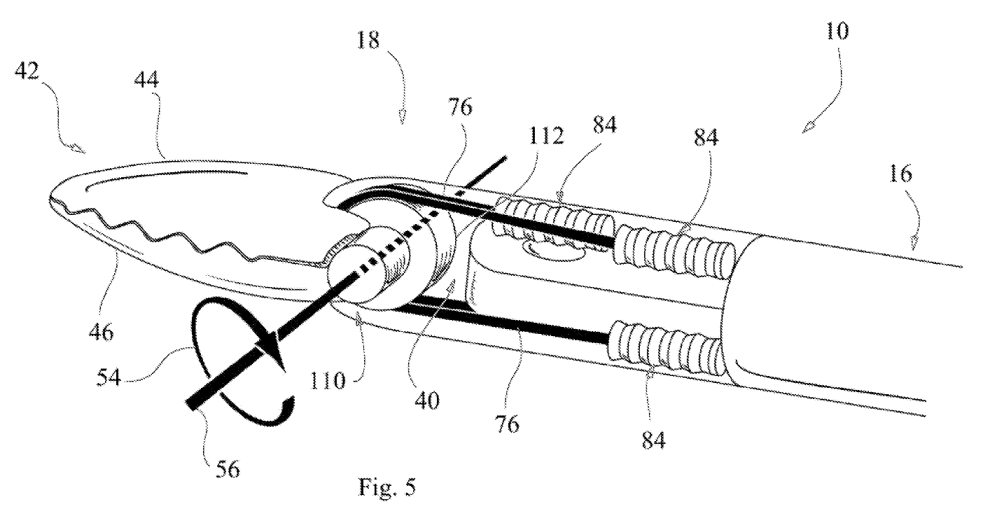

Bellow system 70 is in mechanical connection with end effector 18. Conformational change in shape of bellow 84 causes a mechanical change such as bending, rotation or telescoping of end effector 18. For example, conformational change in shape of either bellow system 70 causes mechanical movement of at least one joint 72 and/or 110 (FIG. 5) of end effector 18.

As illustrated in FIG. 3, expansion or retraction of bellow 84 causes rotation of end effector 18 about yaw joint 72. Yaw joint 72 is illustrated as pulley 74 with suitable mechanical connector 76, such as rope or cable. Each pulley 74 is paired with a couple of bellow systems 70 acting as double actuating cylinders.

As illustrated in FIG. 3, end effector 18 is shown in a neutral orientation. As used herein, the term “neutral orientation” means end effector 18 is substantially aligned with longitudinal axis 36 of shaft 16. End effector 18 in a neutral orientation has not been moved or rotated off to either side, up or down, left or right. With end effector 18 in neutral orientation, each pair of bellow systems 70 are also in neutral positions, not extended or retracted.

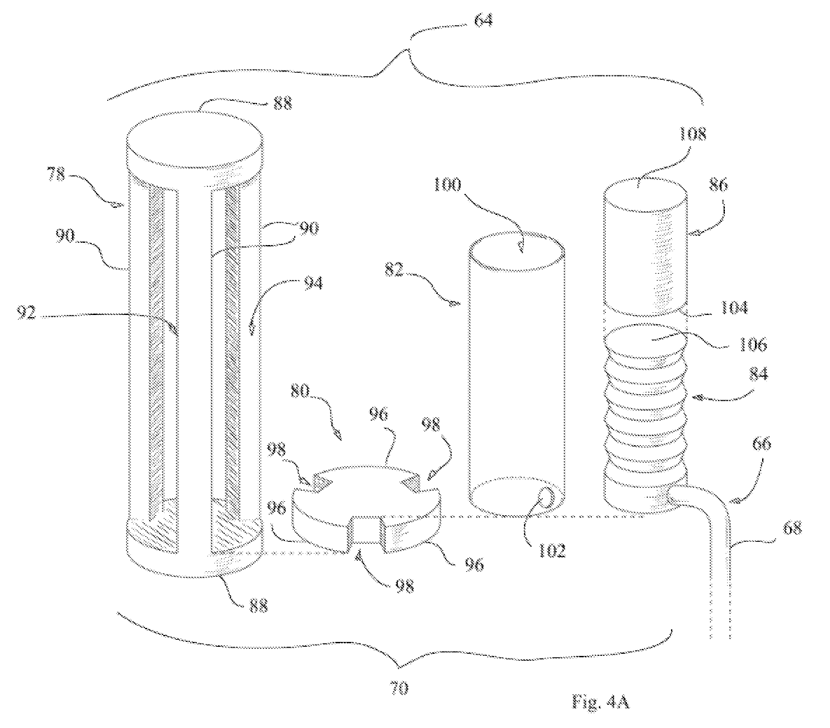

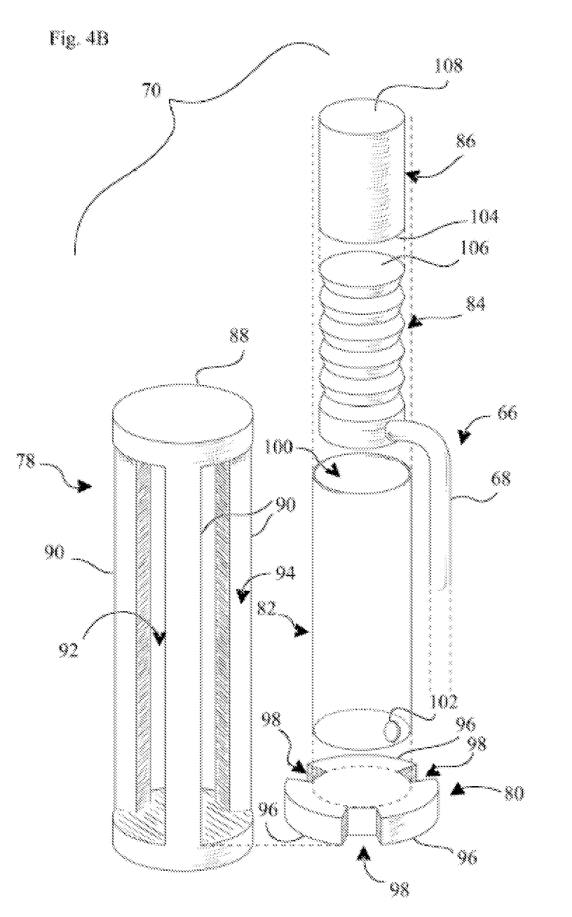

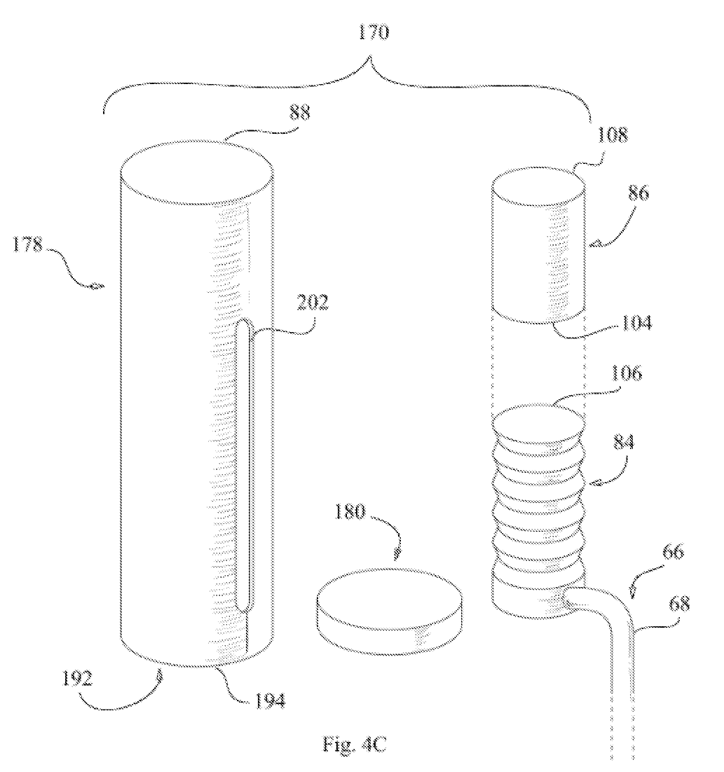

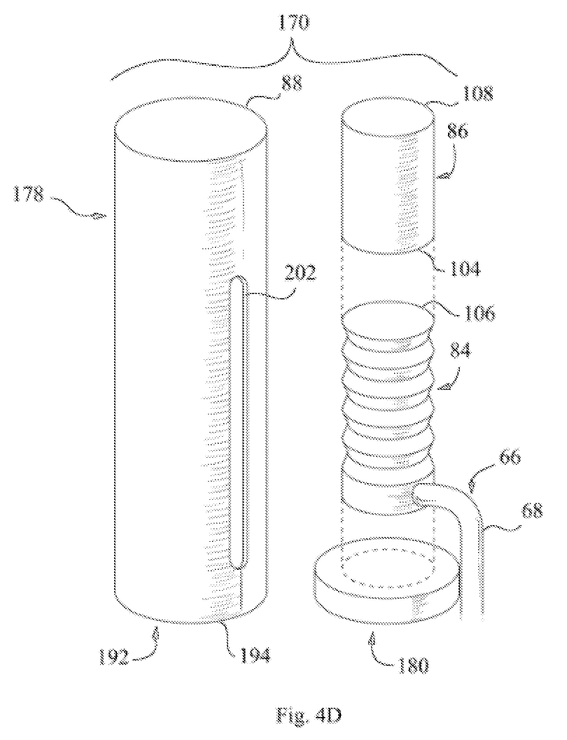

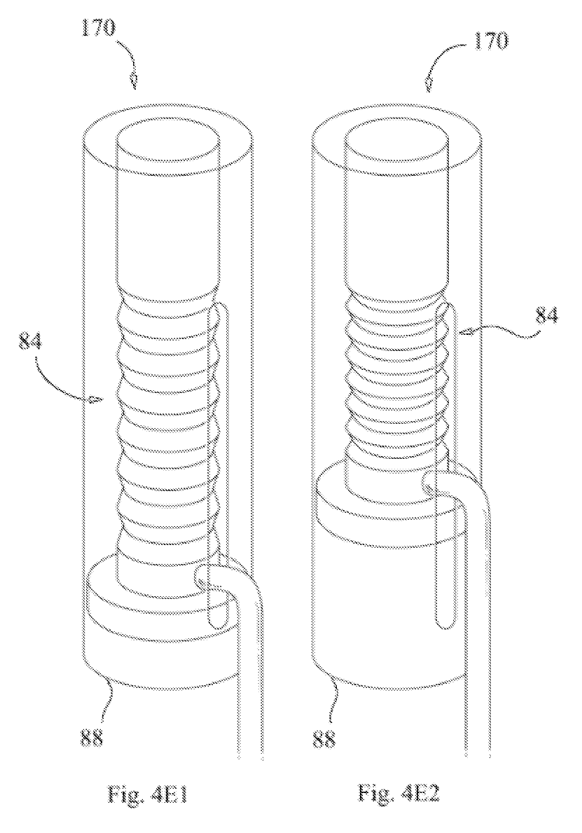

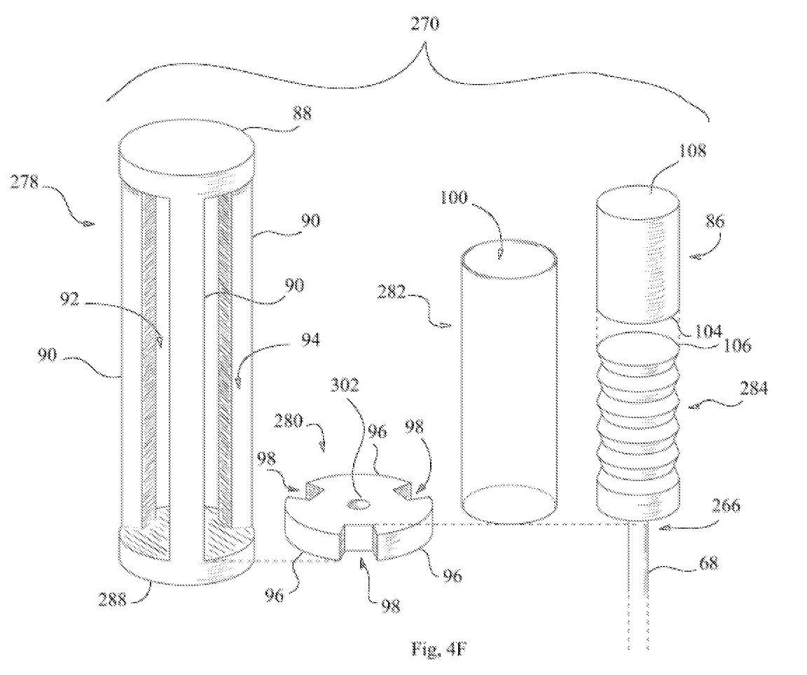

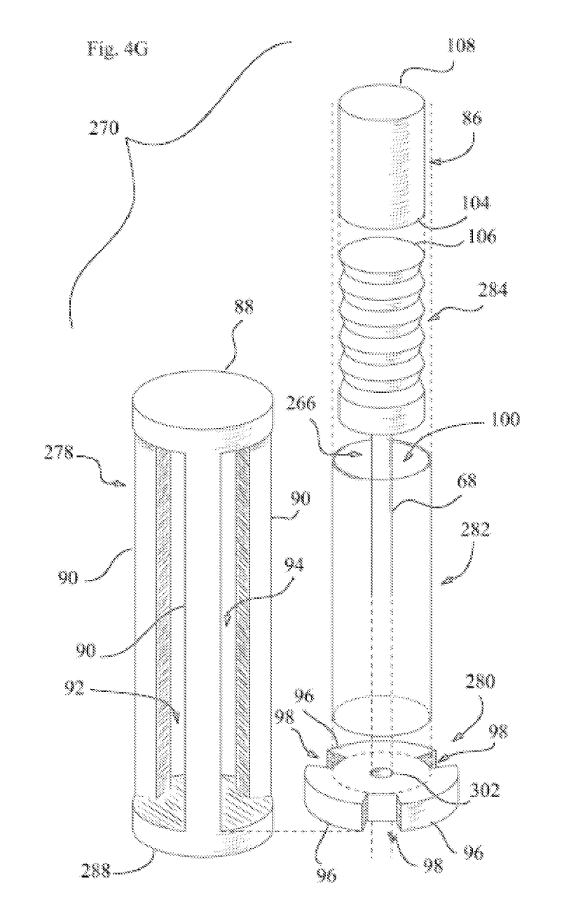

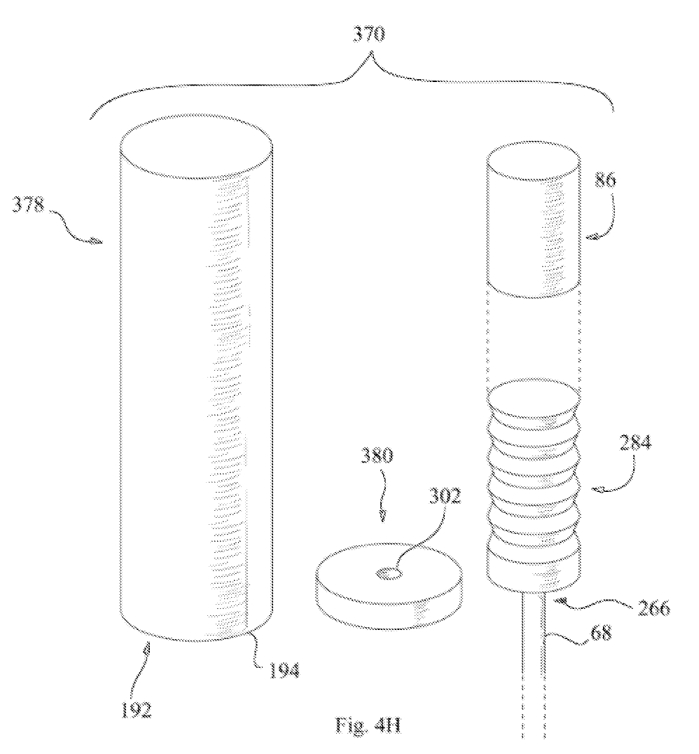

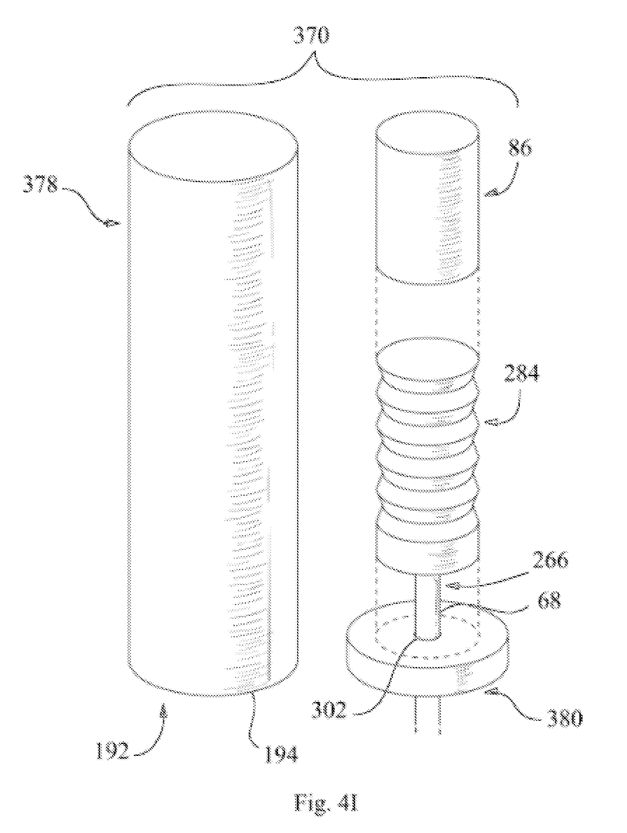

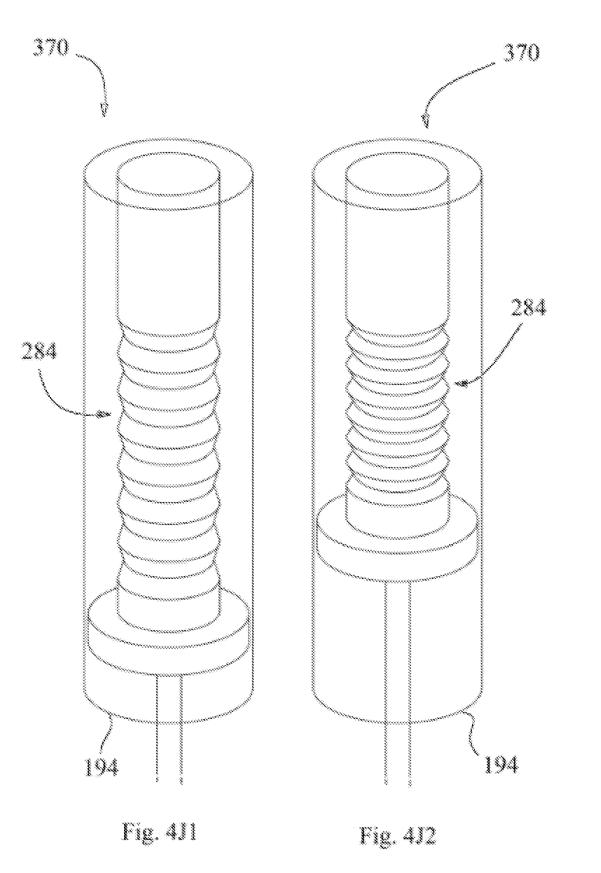

As best illustrated in FIG. 4A, bellow system 70 is shown according to a first embodiment of the present disclosure. Bellow system 70 is shown to include bellow frame 78, sliding mount 80, bellow cylinder 82, bellow 84, and bellow base 86.

Bellow frame 78 includes bellow frame ends 88 and ribs 90. In this illustrative embodiment, there are two bellow frame ends 88 and three ribs 90. However it is envisioned that there could be any number of either bellow frame ends 88 or ribs 90. Bellow frame 78 also defines bellow cavity 92 and bellow frame openings 94.

Sliding mount 80 is generally disk shaped and configured to reside and slideably move within bellow cavity 92. Sliding mount 80 includes sliding mount projections 96 which are configured to correspond with bellow frame openings 94. Sliding mount 80 also defines sliding mount recesses 98 which are configured to correspond with ribs 90 of bellow frame 78.

Bellow cylinder 82 defines bellow cylinder cavity 100 which is configured to hold at least a portion of bellow 84. Bellow cylinder 82 also defines bellow cylinder aperture 102 which provides access to fluid communication for bellow 84 by fluid wire 66. Bellow base end 104 is configured to abut bellow end 106. Bellow base end 108 is configured to abut at least one bellow frame end 88.

As best illustrated in FIG. 4B, assembly of bellow system 70 is shown according to a first embodiment of the present disclosure. At least a portion of bellow 84 resides within bellow cavity 92. Fluid wire 66 provides fluid communication to bellow 84 through bellow cylinder aperture 102. Bellow base 86, bellow cylinder 82 including bellow 84 and sliding mount 80 are arranged within bellow cavity 92 of bellow frame 78. Bellow base end 104 abuts bellow end 106. Bellow base end 108 abuts bellow frame end 88. As bellow 84 expands and contracts, bellow frame end 88 moves causing rotation of end effector 18 about yaw joint 72 as best illustrated by FIG. 3.

As best illustrated in FIG. 4C, bellow system 170 is shown according to a second embodiment of the present disclosure. Bellow system 170 is shown to include bellow frame 178, sliding mount 180, bellow 84, and bellow base 86. Bellow frame 178 has a generally cylindrical shape and includes at least one bellow frame end 88. Bellow frame cylinder 178 also defines bellow cylinder slot 202. Bellow frame cylinder 178 also defines bellow cylinder cavity 192 and at least one bellow frame opening 194. Sliding mount 180 is generally disk shaped and configured to slideably mount within bellow cavity 192. Bellow cylinder cavity 192 is configured to hold at least a portion of bellow 84. Bellow cylinder slot 202 provides access to fluid communication for bellow 84 by fluid wire 66. Bellow cylinder slot 202 is illustrated as an elongated opening. However it is envisioned that bellow cylinder slot 202 could take a number of shapes and sizes according to this embodiment of the present disclosure. Bellow base end 104 is configured to abut bellow end 106. Bellow base end 108 is configured to abut at least one bellow frame end 88.

As best illustrated in FIG. 4D, assembly of bellow system 170 is shown according to a second embodiment of the present disclosure. At least a portion of bellow 84 resides within bellow cavity 192. Fluid wire 66 provides fluid communication to bellow 84 through bellow cylinder slot 202. Bellow base 86, bellow 84 and sliding mount 180 are arranged within bellow cavity 192 of bellow frame 178. Bellow base end 104 abuts bellow end 106. Bellow base end 108 abuts at least one bellow frame end 88.

As best illustrated in FIG. 4E1, as bellow 84 expands, bellow frame end 88 moves relative to bellow 84. Bellow frame end 88 movement causes movement of end effector 18 as previously described and as best illustrated in FIG. 3. As best illustrated in FIG. 4E2, as bellow 84 contracts, bellow frame end 88 moves causing movement of end effector 18 as previously described and as best illustrated in FIG. 3.

As best illustrated in FIG. 4F, bellow system 270 is shown according to a third embodiment of the present disclosure. Bellow system 270 according to the third embodiment of the present disclosure is similar to bellow system 70 according to the first embodiment of the present disclosure. Only the differences between bellow systems 70 and 270 are highlighted below. Bellow system 270 is shown to include bellow frame 278, sliding mount 280, bellow cylinder 282, bellow 284, and bellow base 86. In this illustrative embodiment, bellow frame ends 288 of bellow frame 278 defines bellow aperture (not shown). Sliding mount 280 also defines bellow aperture 302. Bellow cylinder 282 does not define a bellow cylinder slot.

As best illustrated in FIG. 4G, assembly of bellow system 270 is shown according to a third embodiment of the present disclosure. At least a portion of bellow 284 resides within bellow cavity 92. Fluid wire 266 provides fluid communication to bellow 284 through bellow apertures 302 and bellow aperture (not shown) of bellow frame end 288.

As best illustrated in FIG. 4H, bellow system 370 is shown according to a fourth embodiment of the present disclosure. Bellow system 370 according to the fourth embodiment of the present disclosure is similar to bellow system 170 according to the second embodiment of the present disclosure. Only differences between bellow system 370 and previously described systems are highlighted below. Bellow system 370 is shown to include bellow frame 378, sliding mount 380, bellow 284, and bellow base 86. Bellow frame 378 defines bellow cylinder cavity 192 and at least one bellow frame opening 194. Sliding mount 380 also defines bellow aperture 302.

As best illustrated in FIG. 4I, assembly of bellow system 370 is shown according to a fourth embodiment of the present disclosure. At least a portion of bellow 284 resides within bellow cavity 192. Fluid wire 266 provides fluid communication to bellow 284 through bellow aperture 302 and bellow opening 194.

As best illustrated in FIG. 4J1, as bellow 284 expands, bellow frame end 88 and bellow opening 194 move causing movement of end effector 18 as previously described and as best illustrated in FIG. 3. As best illustrated in FIG. 4J2, as bellow 284 contracts, bellow frame end 88 and bellow opening 194 move causing movement of end effector 18 as previously described and as best illustrated in FIG. 3.

Distal actuation segment 70 is illustrated in FIG. 5 as bellow 84. However it is envisioned that any embodiment of the plurality of embodiments of bellow systems illustrated in FIGS. 4A-4J2 could be utilized as distal actuation segment 70 in FIG. 3 or bellow 84 in FIG. 5. Bellow 84 undergoes conformational change in shape upon transmission of operator force from proximal compression area (not shown). The conformational change in shape is shown as either expansion (i.e. extension of bellow 84) or retraction (i.e. reduction, crumpling of bellow 84).

Bellow 84 is in mechanical connection with end effector 18. Conformational change in shape of bellow 84 causes a mechanical change such as bending, rotation or telescoping of end effector 18. For example, conformational change in shape of bellow 84 causes mechanical movement of at least one joint axis 36, 56, or 62 (FIG. 2) of end effector 18. As illustrated in FIG. 5, expansion or retraction of bellow 84 causes rotation of end effector 18 about pitch joint 110. Pitch joint 110 is illustrated as a plurality of pulleys 112. Each pulley 112 is paired with a suitable mechanical connector 76, such as rope or cable. Each pulley 112 paired with a couple of bellows 84 acting as double actuating cylinders. Furthermore, lever 52 of handle 50 (FIG. 1) provides operator's force through hydraulic actuation system 64. It is envisioned that lever 52 actuates one paired set of bellows 84 in order to actuate at least one jaw member 46 of end effector 18 relative to the other jaw member 44 of end effector 18.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

What is claimed is:

1. A surgical instrument comprising:
a frame,
a shaft coupled to the frame, the shaft sized to pass through a trocar, the shaft conformable into a plurality of orientations,
an end effector coupled to the shaft, the end effector sized to pass through the trocar, the end effector and shaft providing at least six degrees of freedom to the end effector relative to the frame, and
a hydraulic articulation control system including a control-effector and at least one bellow, the at least one bellow coupled to the control-effector and the end effector, the at least one bellow used to transfer hydraulic force from the control-effector to the end effector.

2. The instrument of claim 1 wherein the at least one bellow is in hydraulic communication with the control-effector.

3. The instrument of claim 1 wherein the at least one bellow is in mechanical connection with the end effector.

4. The instrument of claim 3 wherein the mechanical connection includes at least one pulley.

5. The instrument of claim 4 wherein the at least one pulley is configured to transmit force from the at least one bellow to cause rotational motion of at least one joint axis of the end effector.

6. The instrument of claim 5 further comprising a plurality of bellows, wherein two bellows are coupled to the at least one pulley.

7. The instrument of claim 6 wherein the two bellows are double actuating cylinders.

8. The instrument of claim 7 wherein the two bellows are each in a neutral position when the end effector is in a neutral orientation.

9. A surgical instrument comprising:
a frame,
a shaft including a proximal end coupled to the frame, the shaft sized to pass through a cannula of a trocar, wherein the shaft is bendable along its longitudinal axis,
a end effector coupled to the shaft, the end effector sized to pass through the cannula of the trocar, the end effector including a pitch joint and a yaw joint, the pitch joint providing rotation of the end effector relative to the shaft in a tilt forward or tilt backward motion, the yaw joint providing rotation of the end effector relative to the shaft in a turn left or turn right motion, the shaft including a roll joint, the roll joint providing rotation of the end effector relative to the frame in a tilt side to side motion, and
a hydraulic articulation control system including:
a control-effector disposed within the frame, and
at least one bellow in hydraulic communication with the control-effector, the at least one bellow used to transfer force from the control-effector to the end effector.

10. The instrument of claim 9 wherein the end effector comprises a pair of jaw members.

11. The instrument of claim 10 wherein the pair of jaw members rotate about the pitch joint.

12. The instrument of claim 11 wherein a lever of the control effector actuates at least one jaw member.

13. The instrument of claim 12 wherein the at least one jaw member rotates about the pitch joint independent of the other jaw member.

14. The instrument of claim 9 wherein the pitch joint and the yaw joint are each revolute joints.

15. The instrument of claim 14 wherein the pitch joint provides rotation about a horizontal axis when the end effector is in a neutral orientation.

16. The instrument of claim 15 wherein the yaw joint provides rotation about a vertical axis when the end effector is in a neutral orientation.

17. The instrument of claim 9 wherein the roll joint provides rotation of the end effector about the longitudinal axis of the shaft when the shaft is in a neutral orientation.

18. A method of operating a surgical instrument, wherein the surgical instrument comprises a frame, a shaft coupled to the frame, a hydraulic control system including a control-effector, an end effector, and at least one bellow in fluid communication with the control-effector, the at least one bellow in mechanical connection with the end effector, the method comprising the steps of:
applying a force to the control-effector,
transferring hydraulic fluid from the control-effector to the at least one bellow,
transferring force from the at least one bellow to the end effector, and
causing at least a portion of the end effector to rotate along at least one of a pitch joint or a yaw joint.

19. The method claim of 18 further comprising the step of rolling the frame causing at least a portion of the end effector to rotate about the longitudinal axis of the shaft.

20. The method claim of 18 wherein the end effector includes a pair of jaw members and further comprising the step of providing force to a lever on the frame causing at least one jaw member to rotate about the pitch joint independent of the other jaw member.
